(19) United States
(12) Patent Application Publication
(10) Pub. No.: US 2025/0302318 A1
(43) Pub. Date: Oct. 2, 2025

(54) SYSTEM AND METHOD FOR NON-CONTACT HEART RATE ESTIMATION

(71) Applicant: Arizona Board of Regents on behalf of Arizona State University, Scottsdale, AZ (US)

(72) Inventors: Yu Rong, Tempe, AZ (US); Arindam Dutta, Tempe, AZ (US); Hanguang Yu, Tempe, AZ (US); Alex Chiriyath, Tempe, AZ (US); Daniel Bliss, Phoenix, AZ (US)

(21) Appl. No.: 18/863,296

(22) PCT Filed: May 19, 2023
(86) PCT No.: PCT/US2023/023021

§ 371 Date: Nov. 5, 2024

Related U.S. Application Data
(60) Provisional application No. 63/344,304, filed on May 20, 2022.

(51) Int. Cl. A61B 5/024; A61B 5/00; A61B 5/05

(52) U.S. Cl. CPC A61B 5/024; A61B 5/05; A61B 5/7225; A61B 5/7235; A61B 5/7271

(57) ABSTRACT

A system and method for non-contact heart rate estimation. In some embodiments, the method includes filtering a radar range history with a first filter, to form a first filtered radar range history; and forming a first estimate of the heart rate of a subject based on the first filtered radar range history, the first filter having a passband including a frequency greater than 20 Hs.

### FIELD

One or more aspects of embodiments according to the present disclosure relate to heart rate estimation, and more particularly to a system and method for non-contact heart rate estimation.

### BACKGROUND

Heart rate estimation has important implications for human health, where robustness and accuracy is of utmost importance, especially in a medical and emergency setting.

It is with respect to this general technical environment that aspects of the present disclosure are related.

### SUMMARY

According to an embodiment of the present disclosure, there is provided a method, including: filtering a radar range history with a first filter, to form a first filtered radar range history; and forming a first estimate of the heart rate of a subject based on the first filtered radar range history, the first filter having a passband including a frequency greater than 20 Hz.

In some embodiments, the first filter is a bandpass filter having a lower band edge of 20 Hz or more, and an upper band edge of 200 Hz or less.

In some embodiments, the forming of the first estimate of the heart rate based on the first filtered radar range history includes calculating a spectrogram of the first filtered radar range history, and estimating the heart rate based on the spectrogram.

In some embodiments, the estimating of the heart rate based on the spectrogram includes counting a plurality of peaks in a portion of the spectrogram, each of the peaks being an S1 peak or an S2 peak.

In some embodiments, the method further includes forming a second estimate of the heart rate of the subject based on the first filtered radar range history.

In some embodiments, the forming of the second estimate of the heart rate includes calculating a power spectrum of the first filtered radar range history.

In some embodiments, the forming of the second estimate of the heart rate further includes finding a peak in the power spectrum.

In some embodiments, the method further includes filtering the radar range history with a second filter to form a second filtered radar range history, the second filter having a passband including a frequency between 0.2 Hz and 5 Hz.

In some embodiments, the method further includes filtering the second filtered radar range history with a third filter to form a third filtered radar range history.

In some embodiments, the method further includes forming a third estimate of the heart rate of the subject based on the third filtered radar range history.

In some embodiments, the method further includes adjusting the third filter based on the first filtered radar range history.

In some embodiments, the adjusting of the third filter based on the first filtered radar range history includes: calculating a power spectrum of the first filtered radar range history; finding a peak in the power spectrum; forming a second estimate of the heart rate, based on the peak; and adjusting the third filter based on the second estimate of the heart rate.

In some embodiments, the method further includes forming a fourth estimate of the heart rate based on the first estimate of the heart rate and the third estimate of the heart rate.

In some embodiments, the fourth estimate of the heart rate is a weighted average of the first estimate of the heart rate and the third estimate of the heart rate.

In some embodiments: the forming of the first estimate of the heart rate based on the first filtered radar range history includes calculating a spectrogram of the first filtered radar range history; and counting a plurality of peaks in a portion of the spectrogram, each of the peaks being an S1 peak or an S2 peak.

According to an embodiment of the present disclosure, there is provided a system, including: a processing circuit; and memory, operatively connected to the processing circuit and storing instructions that, when executed by the processing circuit, cause the system to perform a method, the method including: filtering a radar range history with a first filter, to form a first filtered radar range history; and forming a first estimate of the heart rate of a subject based on the first filtered radar range history, the first filter having a passband including a frequency greater than 20 Hz.

In some embodiments, the first filter is a bandpass filter having a lower band edge of 20 Hz or more, and an upper band edge of 200 Hz or less.

In some embodiments, the forming of the first estimate of the heart rate based on the first filtered radar range history includes calculating a spectrogram of the first filtered radar range history, and estimating the heart rate based on the spectrogram.

In some embodiments, the estimating of the heart rate based on the spectrogram includes counting a plurality of peaks in a portion of the spectrogram, each of the peaks being an S1 peak or an S2 peak.

In some embodiments, the method further includes forming a second estimate of the heart rate of the subject based on the first filtered radar range history.

### BRIEF DESCRIPTION OF THE DRAWINGS

These and other features and advantages of the present disclosure will be appreciated and understood with reference to the specification, claims, and appended drawings wherein:

FIG. 1 is a block diagram of a system and method for non-contact heart rate estimation, according to an embodiment of the present disclosure;

FIG. 2A is a graph of a radar range history, according to an embodiment of the present disclosure;

FIG. 2B is a graph of a filtered radar range history, according to an embodiment of the present disclosure;

FIG. 2C is a graph of a spectrum, according to an embodiment of the present disclosure;

FIG. 3A is a graph of a filtered radar range history, according to an embodiment of the present disclosure;

FIG. 3B is a graph of a spectrum, according to an embodiment of the present disclosure; and

FIG. 4 shows graph of two spectra, with overlaid frequency ranges of bandpass filters, according to an embodiment of the present disclosure.

### DETAILED DESCRIPTION

The detailed description set forth below in connection with the appended drawings is intended as a description of exemplary embodiments of a system and method for non-contact heart rate estimation provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

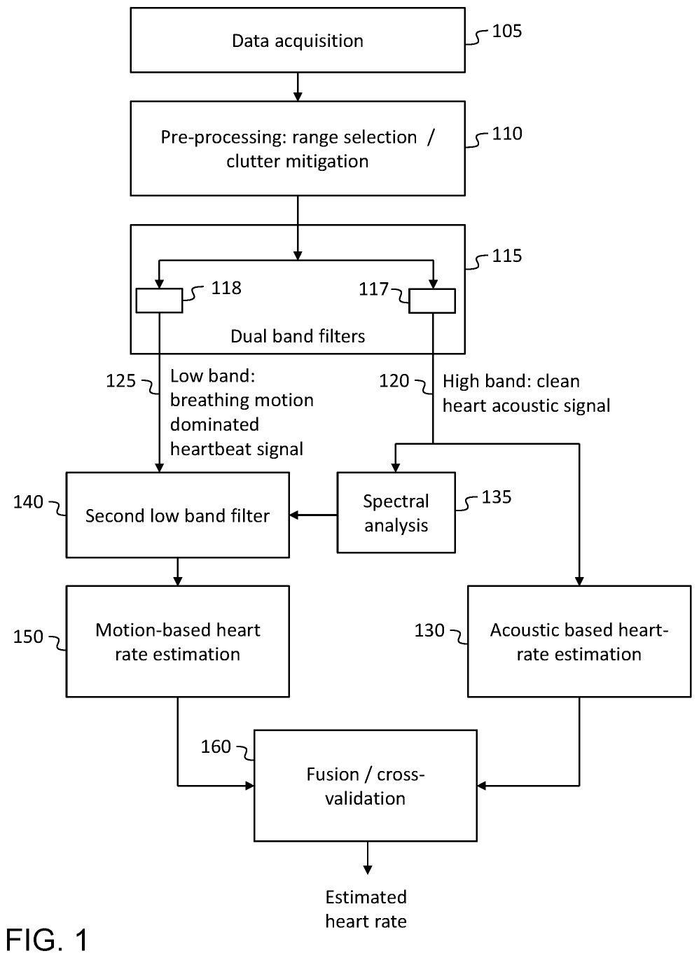

FIG. 1 is a block diagram of a radar system that may be used to estimate the heart rate of a subject (e.g., a human or other animal) based on radar reflections obtained from the skin of the subject (e.g., the skin of the subject's chest). In some embodiments, a data acquisition circuit 105 may be configured to obtain raw data, e.g., raw radar data from a subject. For example, a radar transmitter may illuminate the chest of the subject with radar radiation (e.g., microwave radiation), and a radar receiver may receive reflections from the subject. The radar transmitter and the radar receiver may share an antenna, or the radar transmitter may include a transmitting antenna and the radar receiver may include a receiving antenna. In an embodiment with separate transmitting and receiving antennas, the receiver may be active while the transmitter is transmitting.

The data acquisition circuit 105 may include, for example, a transmitter, which may include a radar pulse generator (which may generate the waveform or waveforms to be transmitted as radar pulses), a power amplifier, for amplifying the pulses, a transmitter matching network, and a transmitting antenna for transmitting the radar pulse. The pulse repetition rate may be, for example, at least twice the highest frequency used in subsequent processing (discussed in further detail below); for example, the pulse repetition rate may be 500 Hz. The data acquisition circuit 105 may further include a receiver which may include a receiving antenna for receiving the radar returns, a receiver matching network, a low-noise amplifier, a downconverter (for converting the received pulses to baseband in-phase and quadrature signals), and one or more analog to digital converters for converting the baseband in-phase and quadrature signals to digital samples.

The output of the data acquisition circuit 105 may be fed to a preprocessing block 110, which may perform range selection and clutter mitigation. Clutter mitigation may involve the suppression of signals caused by reflection of radar pulses from stationary objects near (e.g., behind) the subject. A range fast Fourier transform (a range FFT) may be performed to generate a complex range vector. Range selection may involve finding a range bin with maximum received energy (i.e., finding the element of the complex range vector having the greatest magnitude), and setting a nominal range equal to this range bin. The nominal range may be a nominal distance between the antenna or antennas and the skin of the subject. The instantaneous range may vary, within a neighborhood of the nominal range, as a result, for example, of the subject's breathing and heartbeat.

A range estimate (e.g., an estimate of the instantaneous range) may then be made for each radar pulse. For example, the range estimate may be equal to the product of (i) the wavelength of the radar divided by 2 pi, and (ii) the phase of the element, in the complex phase vector, corresponding to the nominal range. The output of the preprocessing block 110 may be a set of range estimates which may be referred to as a range history. Each element of the range history may be an estimate of the range to the skin of the subject at a respective point in time, the point in time corresponding to a respective pulse reflecting from the skin of the subject. As such, the elements of the range history may be separated in time by the pulse repetition period of the radar. Radar pulses may be processed in batches, which may be overlapping or nonoverlapping (and, if nonoverlapping, contiguous or noncontiguous). The elements of the range history may correspond to a batch of radar pulses.

Each of the preprocessing block 110 and the other blocks (except the data acquisition circuit 105) illustrated in FIG. 1 may be implemented, for example, as a method performed by a single processing circuit (e.g., each of these blocks may be implemented as a respective set of instructions, stored in a computer-readable medium, that, when executed by a processing circuit, cause the processing circuit to perform the method of the block). In other embodiments, one or more of the blocks may be implemented otherwise, e.g., as a respective dedicated circuit. For example, a block may be implemented as a separate stored-program computer with instructions, stored in a computer-readable medium, that, when executed by the stored-program computer, cause the processing circuit to perform the method of the block, or as a processing circuit that is or includes a state machine for performing the method.

The output of the preprocessing block 110 may be sent to a dual band filters block 115. The dual band filters block 115 may include two filters, a first filter 117 (which may be referred to as a “high filter” or as a “high band filter”), and a second filter 118 (which may be referred to as a “low filter” or as a “low band filter”). The inputs of these two filters may both be connected to the input of the dual band filters block 115. The outputs of the high filter 117 and the low filter 118 may be connected, respectively, to a high band output 120 (the signal at which may be referred to as a first filtered radar range history), and a low band output 125 (the signal at which may be referred to as a second filtered radar range history). The low band filter may be a bandpass filter with a lower band edge at 0.4 Hz and an upper band edge at 1.5 Hz. In some embodiments the band edges may be selected differently, e.g., the passband (the range of frequencies between a lower band edge and an upper band edge of the bandpass filter) may be any range of frequencies that includes any frequency between 0.2 Hz and 5 Hz.

As used herein, a “lower band edge” of a filter is a frequency at which the magnitude of the transfer function of the filter crosses a threshold, such that immediately below the lower band edge, the magnitude of the transfer function is less than the threshold, and immediately above the band edge, the magnitude of the transfer function is greater than the threshold. As such, a single-pole high-pass filter with a corner frequency of 10 Hz may have a lower band edge of 10 Hz if the threshold is selected to be 3 dB. Similarly, an “upper band edge” of a filter is a frequency at which the magnitude of the transfer function of the filter crosses a threshold, such that immediately below the lower band edge, the magnitude of the transfer function is greater than the threshold, and immediately above the band edge, the magnitude of the transfer function is less than the threshold. As such, a single-pole low-pass filter with a corner frequency of 10 Hz may have an upper band edge of 10 Hz if the threshold is selected to be 3 dB. As another example, a 2-pole bandpass filter with a lower corner frequency of 10 Hz and an upper corner frequency of 100 Hz may have a lower band edge of 10 Hz and an upper band edge of 100 Hz if the threshold is selected to be 3 dB. As used herein, the “passband” of a filter is the range of frequencies between a lower band edge of the filter (if any) and an upper band edge of the filter (if any). As such, for example, the passband of a high pass filter having a lower band edge and lacking an upper band edge may be the set of all frequencies greater than the lower band edge.

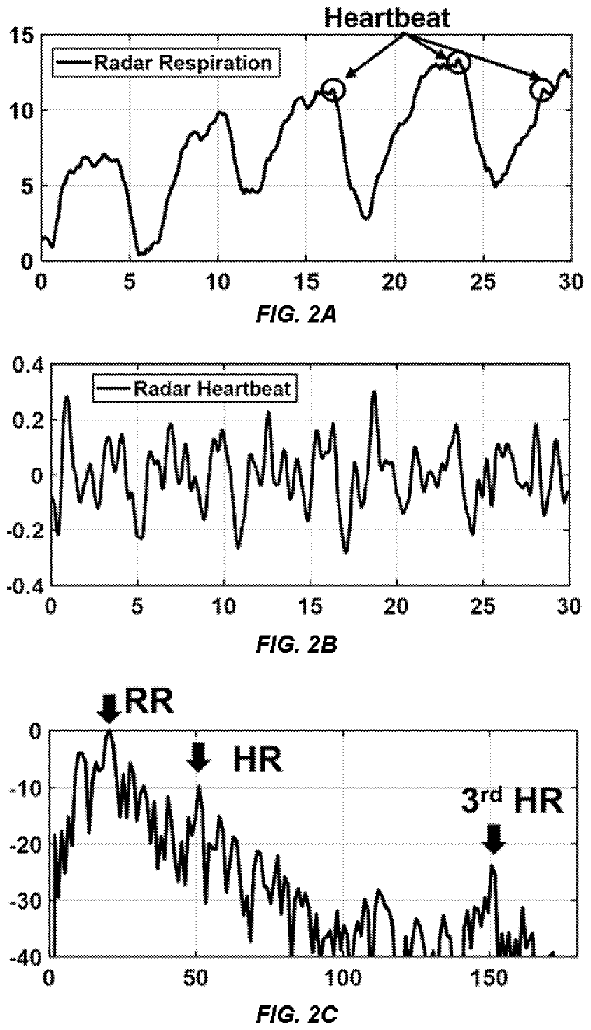

FIG. 2A shows the signal at the input of the dual band filters block 115, in one example, with the horizontal axis having units of seconds, and the vertical axis having units of millimeters (mm). FIG. 2B shows the signal at the low band output 125 of the dual band filters block 115 (i.e., the output of the low band filter 118), in one example, with the horizontal axis having units of seconds, and the vertical axis having units of millimeters (mm). It may be seen that the signal of FIG. 2B does not show an evident periodic signal at the heart rate (50 beats per minute (BPM), in this example); this is borne out by the spectrum of FIG. 2B, which is shown in FIG. 2C, and which lacks a prominent peak at 50 BPM (marked with a marker labeled “HR”). The horizontal axis of FIG. 2C has units of BPM and the vertical axis has units of dB, relative to the largest spectral peak (the peak at the respiration rate, marked with a marker labeled “RR”).

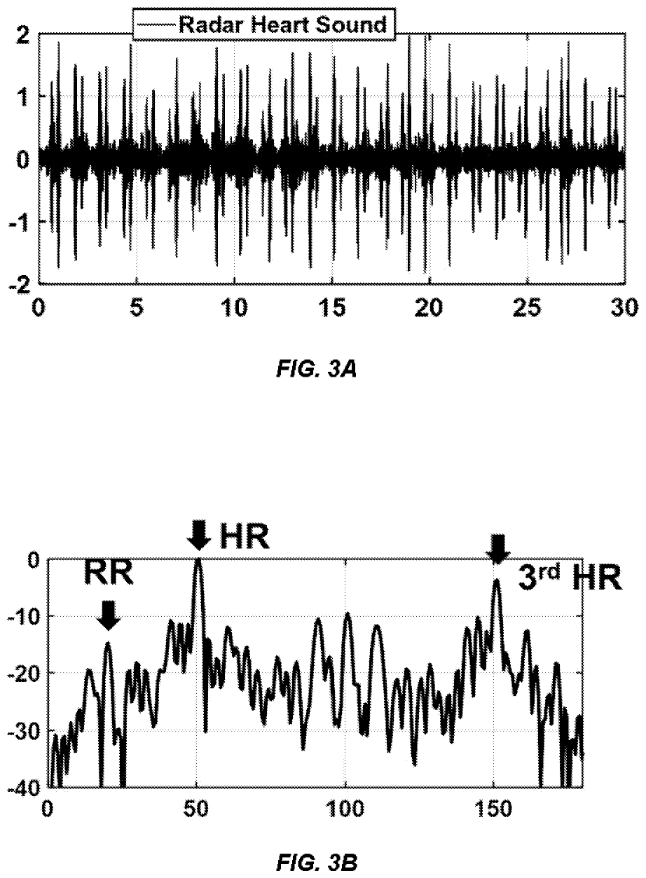

The high band filter 117 may be, for example, a bandpass filter having a passband extending from 50 Hz to 80 Hz. FIG. 3A shows the first filtered radar range history, i.e., the signal at the high band output 120 of the dual band filters block 115, in one example, with the horizontal axis having units of seconds, and the vertical axis having units of microns. In this graph a periodic signal of pairs of pulses (corresponding to the S1 and S2 heart sounds) is readily apparent. The spectrum of the signal of FIG. 3A is shown in FIG. 3B (the horizontal axis of which has units of BPM and the vertical axis of which has units of dB, relative to the largest spectral peak); this spectrum shows a prominent peak at the heart rate (50 BPM, marked with a marker labeled “HR”).

In some embodiments, a first estimate of the heart rate may be formed, in an acoustic based heart-rate estimation block 130 (which analyzes heart sound cycles), based on the first filtered radar range history. For example, a spectrogram of the first filtered radar range history may be formed, and the heart rate may be estimated based on the spectrogram. This may be accomplished, for example, by counting peaks in the spectrogram (using a peak-finding algorithm), each peak being an S1 peak or an S2 peak.

In some embodiments, a second estimate of the heart rate may also be formed, in a spectral analysis block 135, based on the first filtered radar range history. This second estimate of the heart rate may be formed, for example by calculating a power spectrum of the first filtered radar range history, and finding a peak in the power spectrum (e.g., finding the peak marked with a marker labeled “HR” in the power spectrum of FIG. 3B). In such an embodiment, the signal at the low band output 125 may be filtered with a third filter 140 (which may be a second low band filter, and the output of which may be referred to as a third filtered radar range history), the transfer function of which is adjusted (or “adapted”) based on a heart rate estimate which is based on the first filtered radar range history.

For example, the parameters of the second low band filter 140 may be adjusted based on the second estimate of the heart rate and the adjusted second low band filter 140 may then be used to further filter the signal at the low band output 125, to form a third filtered radar range history. For example, the second low band filter 140 may be a narrow band-pass filter (e.g., with a bandwidth of 0.4 Hz), the center frequency of which is adjusted to be equal to the second estimate of the heart rate. This may be accomplished, for example, by implementing the second low band filter 140 as a discrete-time filter (e.g., a finite impulse response (FIR) filter or an infinite impulse response (IIR) filter), the coefficients of which are adjusted based on the second estimate of the heart rate.

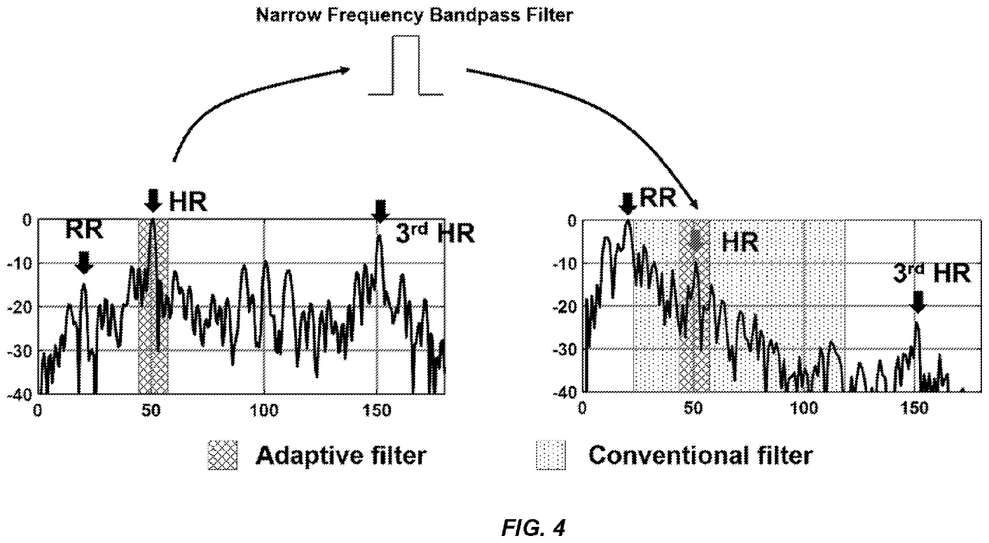

This is illustrated in FIG. 4, in which the graph on the left is the spectrum of FIG. 3B, with a rectangle overlaid over the heart rate peak, showing the passband of the adjusted second low band filter 140. The graph on the right of FIG. 4 is the spectrum of FIG. 2C, with a first shaded rectangle showing the passband of the low band filter 118, and a second shaded rectangle (narrower than the first shaded rectangle) showing the passband of the second low band filter 140. A third estimate of the heart rate may then be obtained, by a motion-based heart rate estimation block 150, which may, for example, calculate a spectrum of the third filtered radar range history, and estimate the heart rate as the frequency of the peak of the spectrum.

The first estimate of the heart rate and the third estimate of the heart rate may be combined in a fusion and cross validation block 160, to form a fourth estimate of the heart rate. In this block, if the difference between the first estimate of the heart rate and the third estimate of the heart rate is less than a threshold (e.g., a threshold between 0 BPM and 5 BPM), then a weighted average of the first estimate of the heart rate and the third estimate of the heart rate may be used as the output of the fusion and cross validation block 160 (and as the fourth estimate of the heart rate). The weights of the weighted average may be chosen based on the difference between the first estimate of the heart rate and the third estimate of the heart rate, with the third estimate of the heart rate being accorded an increasingly small weight for greater differences between the first estimate of the heart rate and the third estimate of the heart rate, and with the third estimate of the heart rate being discarded if the difference between the first estimate of the heart rate and the third estimate of the heart rate is greater than the threshold.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X−Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a system and method for non-contact heart rate estimation have been specifically described and illustrated herein, many modifications and variations will be apparent to those skilled in the art. Accordingly, it is to be understood that a system and method for non-contact heart rate estimation constructed according to principles of this disclosure may be embodied other than as specifically described herein. The invention is also defined in the following claims, and equivalents thereof.

What is claimed is:

1. A method, comprising:
filtering a radar range history with a first filter, to form a first filtered radar range history; and
forming a first estimate of the heart rate of a subject based on the first filtered radar range history,
the first filter having a passband including a frequency greater than 20 Hz.

2. The method of claim 1, wherein the first filter is a bandpass filter having a lower band edge of 20 Hz or more, and an upper band edge of 200 Hz or less.

3. The method of claim 1, wherein the forming of the first estimate of the heart rate based on the first filtered radar range history comprises calculating a spectrogram of the first filtered radar range history, and estimating the heart rate based on the spectrogram.

4. The method of claim 3, wherein the estimating of the heart rate based on the spectrogram comprises counting a plurality of peaks in a portion of the spectrogram, each of the peaks being an S1 peak or an S2 peak.

5. The method of claim 1, further comprising forming a second estimate of the heart rate of the subject based on the first filtered radar range history.

6. The method of claim 5, wherein the forming of the second estimate of the heart rate comprises calculating a power spectrum of the first filtered radar range history.

7. The method of claim 6, wherein the forming of the second estimate of the heart rate further comprises finding a peak in the power spectrum.

8. The method of claim 1, further comprising filtering the radar range history with a second filter to form a second filtered radar range history, the second filter having a passband including a frequency between 0.2 Hz and 5 Hz.

9. The method of claim 8, further comprising filtering the second filtered radar range history with a third filter to form a third filtered radar range history.

10. The method of claim 9, further comprising forming a third estimate of the heart rate of the subject based on the third filtered radar range history.

11. The method of claim 10, further comprising adjusting the third filter based on the first filtered radar range history.

12. The method of claim 11, wherein the adjusting of the third filter based on the first filtered radar range history comprises:
calculating a power spectrum of the first filtered radar range history;
finding a peak in the power spectrum;
forming a second estimate of the heart rate, based on the peak; and
adjusting the third filter based on the second estimate of the heart rate.

13. The method of claim 12, further comprising forming a fourth estimate of the heart rate based on the first estimate of the heart rate and the third estimate of the heart rate.

14. The method of claim 13, wherein the fourth estimate of the heart rate is a weighted average of the first estimate of the heart rate and the third estimate of the heart rate.

15. The method of claim 14, wherein:
the forming of the first estimate of the heart rate based on the first filtered radar range history comprises calculating a spectrogram of the first filtered radar range history; and
counting a plurality of peaks in a portion of the spectrogram, each of the peaks being an S1 peak or an S2 peak.

16. A system, comprising:
a processing circuit; and
memory, operatively connected to the processing circuit and storing instructions that, when executed by the processing circuit, cause the system to perform a method, the method comprising:
filtering a radar range history with a first filter, to form a first filtered radar range history; and
forming a first estimate of the heart rate of a subject based on the first filtered radar range history,
the first filter having a passband including a frequency greater than 20 Hz.

17. The system of claim 16, wherein the first filter is a bandpass filter having a lower band edge of 20 Hz or more, and an upper band edge of 200 Hz or less.

18. The system of claim 16, wherein the forming of the first estimate of the heart rate based on the first filtered radar range history comprises calculating a spectrogram of the first filtered radar range history, and estimating the heart rate based on the spectrogram.

19. The system of claim 18, wherein the estimating of the heart rate based on the spectrogram comprises counting a plurality of peaks in a portion of the spectrogram, each of the peaks being an S1 peak or an S2 peak.

20. The system of claim 16, wherein the method further comprises forming a second estimate of the heart rate of the subject based on the first filtered radar range history.
